(19) United States
(12) Patent Application Publication
(10) Pub. No.: US 2023/0389988 A1
(43) Pub. Date: Dec. 7, 2023

(54) TECHNIQUES AND SYSTEM TO COLLAPSE A GASEOUS PIPELINE IN A LIQUID MEDIUM

(71) Applicant: Lumenis Ltd., Yokneam Illit (IL)

(72) Inventor: Hernan Altman, Tivon (IL)

(73) Assignee: Lumenis Ltd., Yokneam Illit (IL)

(21) Appl. No.: 18/328,149

(22) Filed: Jun. 2, 2023

Related U.S. Application Data
(60) Provisional application No. 63/348,719, filed on Jun. 3, 2022.

(51) Int. Cl. A61B 18/26

(52) U.S. Cl. CPC A61B 18/26; A61B 2018/00577

(57) ABSTRACT

Techniques and a laser system for modulating laser pulses are provided. The laser pulses can be modulated to cause a gaseous pathway formed when the laser pulses are emitted into a liquid medium to collapse within a specified distance. The instantaneous power of the laser pulses can be modulated in an opposite phase from the growth of vapor bubbles forming the gaseous pathway.

### CROSS-REFERENCE TO RELATED APPLICATIONS

This application claims the benefit of priority under 35 U.S.C. § 119 of U.S. Provisional Application No. 63/348,719, filed Jun. 3, 2022, the entire disclosure of which is hereby incorporated by reference.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, the stones are required to be fragmented into smaller pieces. A technology known as laser lithotripsy may be used for such fragmenting processes, wherein for small to medium sized urinary stones, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it. In another instance, a laser and optic fiber technology is used in ablation treatments. During an ablation treatment, laser light is delivered to the tissue to vaporize the tissue or to induce damage within the tissue. Such ablation treatments may be used for treating various clinical conditions, such as Benign Prostate Hyperplasia (BPH), or cancers, such as bladder cancer.

It has been shown in clinical usage that use of lasers in lithotripsy procedures may cause unintentional damage to surrounding tissue, such as the ureter. Accordingly, there is a risk associated with using lasers in lithotripsy and other procedures.

### BRIEF SUMMARY

The present disclosure provides medical devices and techniques for enhancing safety of laser beam usage in clinical procedures. In particular, the present disclosure is directed at enhancing safety of laser beam usage in a liquid environment. Many treatment procedures are carried out in a liquid environment. Liquid environments tend to absorb a significant portion of laser energy. For example, the liquid medium can absorb and attenuate the laser energy, leaving less energy available for the desired surgical effect on the target. Additionally, although energy absorbed by the liquid medium can protect surrounding tissue by limiting the range of energy transfer, it can also heat surrounding tissue, leading to unwanted side effects or potential safety issues. The present disclosure provides to control the present or absence of liquid (e.g., based on modulating a vapor tunnel, which is described in detail below) to control the range of the laser beam.

Further, the need to irrigate the areas with cooling fluids can be increased when using lasers. Such safety issues can be further complicated based on the type of laser used in the procedure. For example, Thulium fiber lasers (TFL) are one laser modality that is employed in lithotripsy procedures. Damage from TLF energy emissions has been clinically observed even though absorption in a liquid medium of laser energy emitted by TFL is higher (e.g., on the order of four (4) times higher) than other laser mediums used in lithotripsy (e.g., Holmium). As a specific example the expected mean travel distance in water of light emitted by a Thulium laser is 0.1 millimeters (mm) while the expected mean travel distance in water of light emitted by a Holmium laser is 0.4 mm.

This is partly because lasers, such as TFL, which deliver longer pulses relative to some other laser modalities (e.g., due to peak power limitations, or the like) may result in energy delivery to relatively larger distances. More particularly, as TFL lasers have longer pulses relative to other modalities, a more elongated vapor tunnel is formed in the liquid environment leading to greater range of the energy delivery. This effect is independent of the wavelength absorption in the liquid medium owing to the creation of a “vapor tunnel.” During activation of the laser, the interaction between the laser energy and the liquid medium forms a gaseous pathway (or vapor tunnel) from a bubble or a chain of bubbles in the liquid medium. This gaseous pathway has an absorption coefficient that is smaller than the absorption coefficient of the liquid medium, thereby facilitating a greater amount of the laser energy to pass from a laser fiber tip to the target. Additionally, the longer pulses of a TFL create a more extensive area of thermal damage (e.g., due to the relatively longer exposure time of the tissue as compared to the tissue's thermal relaxation constants). It is to be appreciated that a shorter pulse will in general create a more localized damage, with more localized thermal dissipation area thus smaller thermal lesion.

Accordingly, using TFL (or any laser generating very long pulses) for lithotripsy procedures creates an increased potential safety risk. There are several factors that can elevate this safety risk further. For example, the safety risk may be elevated when operating near sensitive areas (e.g., the ureter) or when operating with limited visibility (e.g., such as during lithotripsy due to the turbid media created during stone breaking. As another example, the safety risk may be elevated when working at high repetition rates or when the target is mobile (e.g., such as where the stone moves due to retropulsion, irrigation, or other factors).

The present disclosure provides a laser system where the laser pulse is modulated such that the vapor bubble created by the laser pulse is intentionally collapsed to shorten the gaseous pathway to reduce the reach (or propagation) of the laser energy from the fiber tip. Said differently, the present disclosure provides apparatus and methods to modulate a laser pulse to avoid resonant generation of a gaseous pathway (or vapor bubble) beyond an intended target.

In specific embodiments, the laser pulse is modulated at an opposite phase of that of the bubble. As another specific embodiment, the distance from the tip of the fiber to the target is determined (e.g., estimated, measured, or the like) and the laser pulse is modulated to achieve growth of the gaseous pathway to the determined distance. As a further example, the laser pulse is modulated such that the gaseous pathway is greater than or equal to the determined distance minus a threshold distance and less than or equal to the determined distance plus the threshold distance. Modulation of the laser pulse can be repeated in cycles to continue the treatment while maintaining (e.g., growing or collapsing) the gaseous pathway such that the gaseous pathway is within a threshold from an intended distance.

With some embodiments, the power of the laser pulse can be reduced based on a change in the distance between the tip of the fiber and the target of greater than or equal to a threshold level. As a further embodiment, the power of the laser pulse can be reduced based on a change in the distance between the tip of the fiber and the target of greater than or equal to a threshold level within a specified time period. For example, a sudden increase in the distance between the fiber tip and the target may indicate the actual target (e.g., the stone, or the like) has moved and the fiber is now pointed directly at tissue behind where the stone was. In another example, a system capable of differentiating between stone and soft tissue may collapse the vapor tunnel when tissue is detected.

These and other embodiments of the present disclosure will be readily apparent from the following detailed description.

### BRIEF DESCRIPTION OF THE SEVERAL VIEWS OF THE DRAWINGS

Non-limiting embodiments of the present disclosure are described by way of example with reference to the accompanying figures, which are schematic and not intended to be drawn to scale. It is to be appreciated that various figures included in this disclosure may omit some components, illustrate portions of some components, and/or present some components as transparent to facilitate illustration and description of components that may otherwise appear hidden. For purposes of clarity, not every component is labelled in every figure, nor is every component of each embodiment shown where illustration is not necessary to allow those of ordinary skill in the art to understand the disclosure. In the figures:

FIG. 1 illustrates a plot describing a gaseous pathway, in accordance with at least one embodiment.

FIG. 2 illustrates another plot describing a gaseous pathway, in accordance with at least one embodiment.

FIG. 3 illustrates a system, in accordance with at least one embodiment.

FIG. 4 illustrates a treatment environment, in accordance with at least one embodiment.

FIG. 5 illustrates a method, in accordance with at least one embodiment.

FIG. 6 illustrates another method, in accordance with at least one embodiment.

FIG. 7 illustrates another method, in accordance with at least one embodiment.

FIG. 8 illustrates a block diagram of a computing environment, in accordance with at least one embodiment.

### DETAILED DESCRIPTION

The foregoing has broadly outlined some features and technical advantages of the present disclosure, such that the following detailed description of the disclosure may be better understood. It is to be appreciated by those skilled in the art that the embodiments disclosed may be readily utilized as a basis for modifying or designing other structures for carrying out the same purposes of the present disclosure. For instance, components and features disclosed hereby may be selectively combined without departing from the scope of this disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the claims. Further, although various embodiments may be described with respect to laser lithotripsy or laser ablation treatments, reference to these conditions should not be construed as limiting the possible applications of the disclosed aspects. For example, the disclosed aspects may be utilized for treating clinical conditions such as, cancers (e.g., liver cancer, lung cancer, and the like) or cardiac conditions (e.g., by ablating and/or coagulating a part of the tissue in the heart).

It is to be appreciated that control of the gaseous pathway is complicated by the complex nature of the dynamics and harmonics of bubbles formed in the liquid medium along with differences between energy emissions from different types of lasers. To that end, a discussion of laser pulses and bubble formation is provided prior to describing the techniques for modulating the laser pulse to intentionally collapse the bubble.

In some embodiments of the present disclosure, a laser with low peak-power and long pulses, such as a TFL, may be utilized so that the total size and stability of the bubble cascade generated is much longer (along the fiber axis from the laser fiber tip to the target) than the single bubble of a typical short pulse laser. Therefore, and unexpectedly, even though Thulium (Tm) photons have much stronger absorption in water than Holmium (Ho), when comparing short pulse Ho to long pulse Tm, the latter travels further as it travels through the longer train of bubbles produced by the TFL. Further, a longer pulse duration takes better advantage of the cascade of bubbles as the pulse persists while the bubbles reach the target. While it has been observed that the TFL pulses are severed into several sub-pulses due to the multiple bubble collapses, which often results in a widely variable energy delivery during the pulse duration, the Ho laser energy may produce similar overall energy delivery.

As mentioned above, the bubble has its own dynamics, and once initiated, in many aspects it is no longer related to the characteristics of the laser pulse. An initial bubble formed by firing the laser first grows to a maximum size (dependent on the initial peak power of the pulse), and then collapses. The typical bubble duration for a Ho laser is approximately 200 to 300 microseconds (μs). Laser pulses which are longer than the life cycle of the bubble, create several bubbles, in a series, emanating from the laser fiber tip towards the target.

Infrared lasers, such as Ho lasers (having a wavelength of approximately 2100 nanometers (nm) and Tm lasers (having a wavelength of approximately 1940 nm to 2010 nm) are strongly absorbed in a liquid environment. For example, the photons at 1940 nm wavelength have an absorption coefficient of 110 centimeter (cm)⁻¹ and the photons at 2100 nm wavelength have an absorption coefficient of 25 cm⁻¹. Typically, in a liquid environment, many of the photons produced by the Ho lasers are absorbed before the laser pulse travels a distance of 0.5 mm from where it exits the optical fiber while the photons produced by the Tm lasers are absorbed before the laser pulse travels a distance of 0.1 mm from where it exits the optical fiber. Along the same lines, a solid-state Ho laser is characterized by high peak power and relatively short pulses, while a Tm fiber laser (TFL) is characterized by a lower peak power. Therefore, it takes a longer time for a TFL, compared to a solid-state Ho laser, to generate an equal amount of energy. For example, a TFL will generate an energy of 0.5 Jules in a pulse duration of about 1 millisecond (ms) while it takes about 0.2 ms for a solid-state Ho laser to generate the same amount of energy. As will be further discussed below, it has been observed that the life cycle of a single bubble lasts about 0.2 to 0.3 ms. Moreover, it has been observed that the bubble, once initiated, has its own dynamic characteristics which is in many aspects unrelated to the laser pulse (except for the pulse's initial characteristics at a very short time frame of a few tens of microseconds). Based on the above, a short solid-state Ho laser pulse may end before the bubble will reach its maximum size while a longer TFL pulse will last 3-4 times the life of the bubble.

As introduced above, a laser pulse that is longer than the life cycle of a single bubble results in the creation of a cascade of multiple bubbles. A cascade of multiple bubbles may create a gaseous pathway to the target. However, such a bubble cascade is characterized by sequence of independent bubbles, which expand and collapse at different times and at different locations along the pathway. As a result, the effective length of the gaseous pathway changes over time.

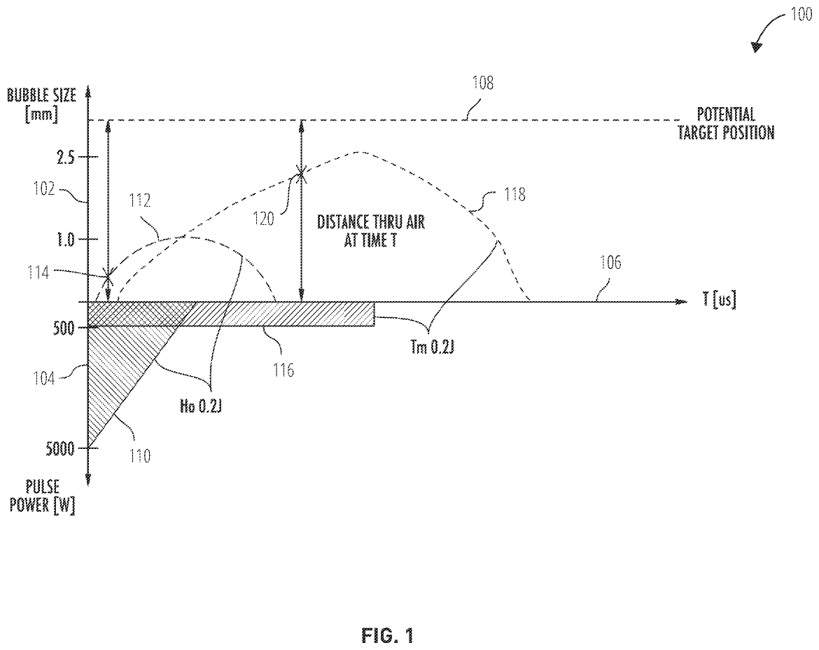

FIG. 1 illustrates an exemplary diagram 100 of pulses of a Ho laser and a Tm laser according to one or more embodiments described herein. In the illustrated embodiment, the Ho and Tm lasers have an equal energy of 0.2 Joules (J). Diagram 100 illustrates a typical high peak-power Ho laser short pulse versus low peak-power quasi-continuous Tm long pulse. The diagram 100 includes bubble size (in mm) on the positive y-axis 102 (with a potential target position at line 108), pulse power (in Watts (W)) on the negative y-axis 104, and time (in μs) on the x-axis 106. With respect to the high peak-power Ho laser short pulse, area 110 corresponds to the pulse power, curve 112 corresponds to the bubble created, and line 114 corresponds to the effective distance the laser pulse travels through the liquid medium. With respect to the low peak-power quasi-continuous Tm long pulse, area 116 corresponds to the pulse power, curve 118 corresponds to the bubble created, and line 120 corresponds to the effective distance the laser pulse travels through the liquid medium.

In both cases, after a short delay, on the order of a few tens of microseconds (μs), following the laser pulse initiation, a bubble is created and starts to expand. In both cases, the life cycle of the bubble, once created, appears to have its own internal dynamics which are not solely related to the laser pulse, and includes the internal vapor pressure generated in the bubble enucleation site, which in turn can depend on absorption, instantaneous peak power, laser beam quality, and/or the presence and amount of air pockets inside the liquid.

It takes time for the bubble to expand. In the case of the Ho pulse, the bubble reaches its maximum size well after the highest power peak of the laser and in the case of the Tm laser, the bubble reaches its maximum size during the laser pulse. Also shown in FIG. 1 is that for an exemplary target distance 112 (positioned at a distance of about 3 mm), the effective distance the Tm laser pulse can travel through the liquid medium (e.g., line 120) is much longer than the effective distance the Ho laser pulse can travel through the liquid medium (e.g., line 114). Several embodiments described herein take advantage of the independent dynamics of the bubbles to intentionally collapse the bubble in order to reduce or define the distance that the laser pulse will travel through the liquid medium. In particular, the present disclosure provides to modulate the laser pulse power with an opposite phase to that of the bubble to intentionally collapse the bubble (or to intentionally collapse the tunnel created by a chain of bubbles) to avoid resonant generation of bubbles beyond the intended target.

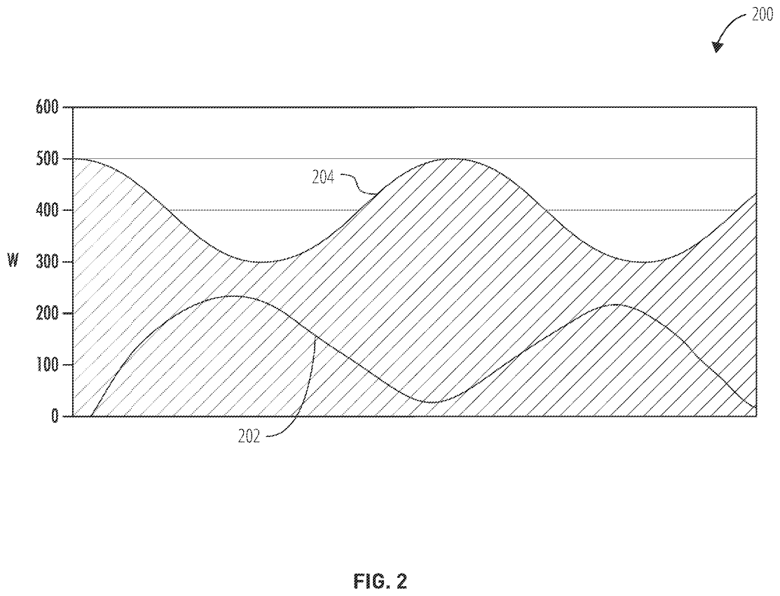

FIG. 2 illustrates an exemplary diagram 200 of laser pulse power modulation 204 in relation to bubble size 202 according to one or more embodiments described herein. In various embodiments, diagram 200 includes an exemplary pulse power modulation scheme. A pulse modulation scheme may include one or more settings, modes, parameters, characteristics, features, and the like of the pulse, the environment (e.g., liquid medium, distance), and/or various components utilized to implement the pulse (see e.g., laser system 300). The pulse power modulation 204 is shown by a sinusoidal-type pattern shown in the slanted hatched area. In this case, power is modulated from a high level of 500 W to a low level of 300 W. According to this aspect of the disclosure, the laser power over long pulses is modulated in an opposite direction to the bubble dynamics described above so that when the bubble expands the laser is modulated down and when the bubble collapses the laser is modulated up. Embodiments are not limited in this context.

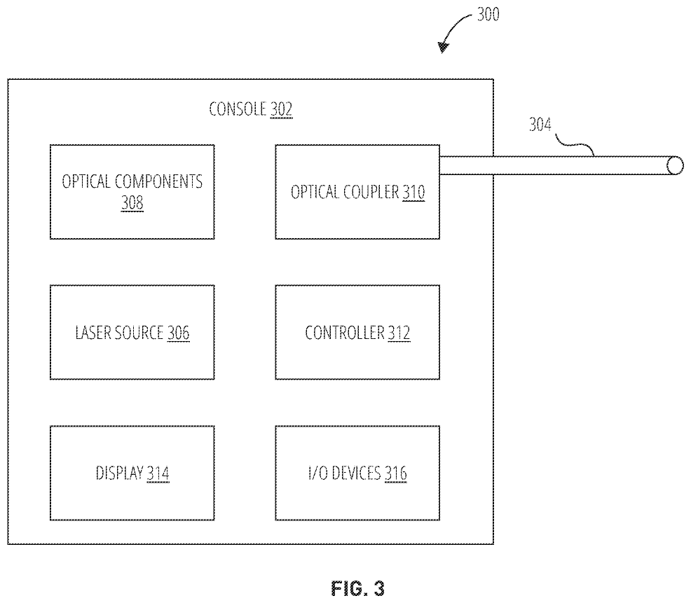

FIG. 3 illustrates a 300 in accordance with non-limiting example(s) of the present disclosure. The laser system 300 includes a console 302 and a fiber optic cable 304. In general, the laser system 300 is arranged to generate laser energy emissions and to modulate the laser emissions to control a size of a bubble when the laser energy is emitted in a liquid medium. For example, the laser system 300 can be implemented to modulate pulses of laser energy such that a vapor bubble created by the laser pulses is intentionally collapsed to shorten a gaseous pathway formed by the vapor bubbles with the intention to reduce the reach (or propagation) of the laser energy emitted from a fiber tip coupled to the laser system 300.

The laser system 300 includes a console 302 and fiber optic cable 304 coupled to the console. It is noted that although the console 302 and components of the console 302 are depicted as arranged in a single housing, some examples may provide multiple housings. The console 302 includes a laser source 306, optical components 308, an optical coupler 310, and a controller 312. Console 302 may further optionally include a display 314 and/or input and/or output (I/O) devices 316.

Although not illustrated in this figure, it is to be appreciated that the laser system 300 may be included in, integrated into, or connected to a treatment laser system, for example, a laser ablation system arranged to perform one or more laser treatment procedures (e.g., lithotripsy, ablation, or the like). In particular, the laser system 300 can be implemented to enhance or increase the safety of the laser treatment procedure.

The laser source 306 may comprise a laser arranged to generate a treatment beam (e.g., a TFL, an Ho laser, or the like). The optical components 308 can include any of a variety of optical components (e.g., polarizers, beam splitters, beam combiners, light detectors, filters, wavelength division multiplexers, collimators, circulators) arranged to couple light emitted from laser source 306 with optical coupler 310 and ultimately fiber optic cable 304. The optical coupler 310 can be arranged to provide an optical coupling between a proximal end of fiber optic cable 304 and the console 302 such that light emitted from the laser source 306 may be transmitted through the fiber optic cable 304 and emitted by the distal end of the fiber optic cable 304.

The controllers 312 can include circuitry such as an application specific integrated circuit (ASIC). As another example, the controller 312 can include a processor and memory storing instructions executable by the processor which when executed cause the laser system 300 to implement the operations described herein. For example, the controller 312 can be arranged to cause laser system 300, and particularly the laser source 306, to modulate pulses emitted by the laser source 306 such that vapor bubbles and gaseous pathways generated when laser energy is emitted from the distal end of the fiber optic cable 304 collapse within a specified distance from the distal end of the fiber optic cable 304.

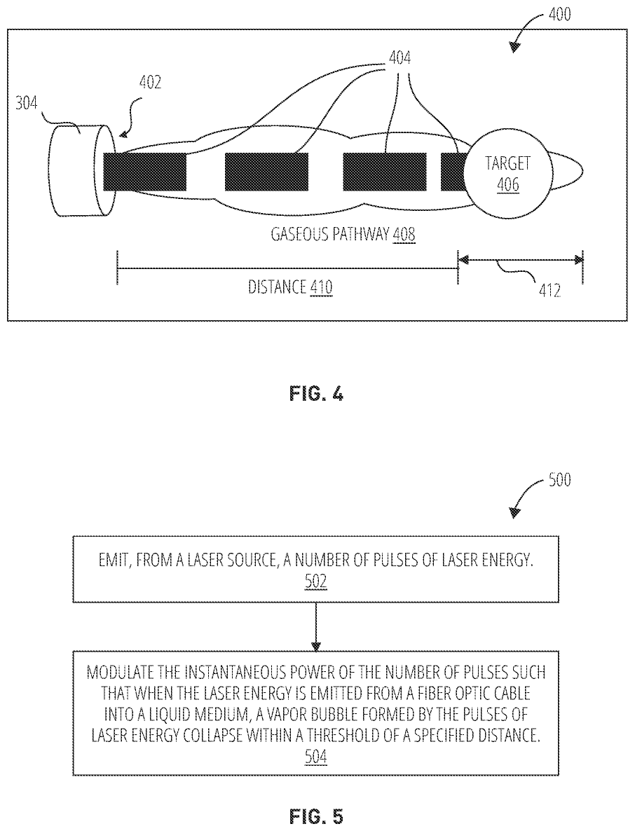

Additionally, in some embodiments, the optical components 308 can include an optical receiver arranged to measure a quantity of light reflected from the laser system 300 (e.g., by the input facet to the fiber optic cable 304, by the output facet of the fiber optic cable 304, by the (see FIG. 4), or the like. As will be explained in more detail herein, the laser system 300 can measure a distance between a distal end of the fiber optic cable 304 and a target and use the measured distance to control the distance with which the gaseous pathway (see FIG. 4) collapses. That is, the controller 312 can send control signals to the laser source 306 to cause the laser source 306 to modulate an instantaneous power of generated laser pulses based on the measured distance (e.g., to cause the gaseous pathway to collapse) within a threshold distance of the measured distance.

The display 314 can include any of a variety of displays (e.g., LCD displays, LEDs, or the like) arranged to provide a visual indication (e.g., graphical, a graphical user interface, graphical elements, light sequences, or the like) to communicate information to a user of the laser system 300. The I/O devices 316 can include any of a variety of devices (e.g., mouse, keyboard, joystick, button, pedal, speaker, or the like) arranged to receiving input from a user or provide output to a user. With the display 314 and the I/O devices 316 a user can interact with (e.g., provide inputs to or received output from) the laser system 300 to for example, provide an indication of an intended distance with which the vapor bubble is to be collapsed or an indication of an expected distance with which the vapor bubble will be collapsed.

FIG. 4 illustrates a treatment environment 400. As contemplated herein, treatment environment 400 will be a liquid medium, such as, for example, as may be encountered in a lithotripsy procedure. A distal end 402 of a fiber optic cable 304 is introduced into the treatment environment 400, such as, for example, via a working channel or a ureteroscope (not shown). Laser pulses 404 are emitted from the distal end 402 into the treatment environment 400 towards a target 406. For example, a clinician can “aim” the distal end 402 at the target 406 (via positioning the distal end 402 or the ureteroscope, or the like). The clinician can then activate a laser source (e.g., laser source 306, or the like) to cause the laser pulses 404 to be generated and emitted into the treatment environment 400. As outlined above, the laser pulses 404 will generate vapor bubbles in the treatment environment 400 forming a gaseous pathway 408. The present disclosure provides to modulate the instantaneous power of the laser pulses 404 such that the gaseous pathway 408 collapses at a specified distance. In some examples, the instantaneous power of the laser pulses 404 is modulated at an opposite phase from the growth of the vapor bubbles (not shown) that form the gaseous pathway 408. In some embodiments, the instantaneous power of the laser pulses 404 are modulated such that the gaseous pathway 408 collapses between 1 and 1.3 times the distance between the distal end 402 and the target 406 (e.g., distance 410), which is depicted as a threshold distance 412.

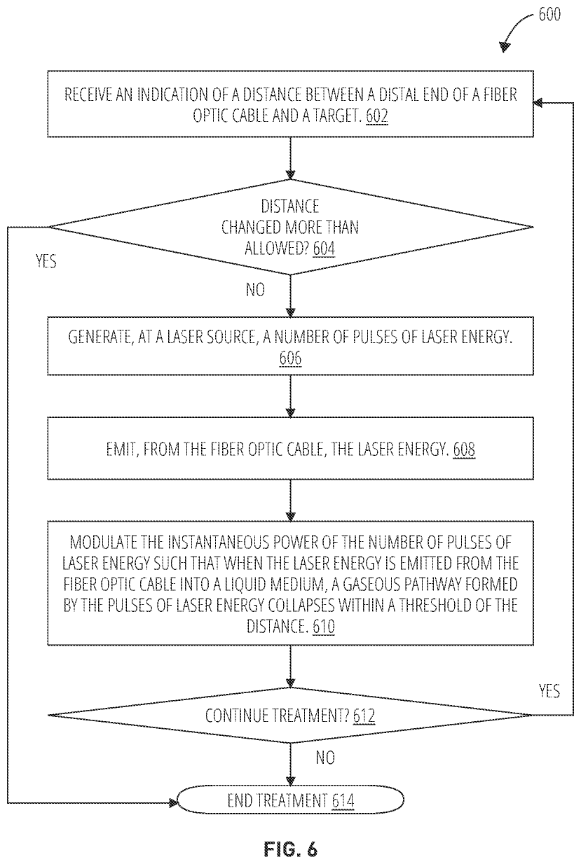

FIG. 5 illustrates a method 500 for modulating laser pulses to control the size of a gaseous pathway created by vapor bubble when the laser pulses are emitted into a liquid medium. Method 500 can be representative of some or all the operations that may be executed by one or more components, devices, or systems described herein, such as one or more portions of laser system 300. Furthermore, method 500 can be implemented by a medical device arranged to emit laser energy into a liquid medium (e.g., treatment environment 400) as part of using the medical device as part of a therapeutic procedure (e.g., lithotripsy). Although the laser system 300 and the treatment environment 400 are used when describing method 500, embodiments are not limited in this context and the method 500 can be implemented by a laser system having a different arrangement to laser system 300 or including different components than shown or described with respect to laser system 300.

As depicted, method 500 may begin at block 502. At block 502 “emit, from a laser source, a number of pulses of laser energy” several pulses of laser energy are emitted from a laser source. For example, laser source 306 may generate pulses of laser energy and cause that the pulses of laser energy be emitted from fiber optic cable 304 (e.g., as laser pulses 404, or the like).

Continuing to block 504 “modulate the instantaneous power of the number of pulses such that when the laser energy is emitted from a fiber optic cable into a liquid medium, a vapor bubble formed by the pulses of laser energy collapse within a threshold of a specified distance” the instantaneous power of the laser pulses generated by the laser source (e.g., at block 502) can be modulated to cause a vapor bubble that is formed when the laser pulses are emitted into a liquid medium to collapse within a specified distance. For example, controller 312 can send signals (e.g., control signals, information elements, or the like) to laser source 306 to cause laser source 306 to modulate the instantaneous power of the laser pulses generated (e.g., laser pulses 404) to cause the gaseous pathway 408 to collapse within threshold distance 412.

It is to be appreciated, that as used herein, the instantaneous power of the laser pulses generated by the laser source can be modulated either up (e.g., increase power) or down (e.g., decrease power) to increase or decrease the length of the vapor bubble. Furthermore, it is to be appreciated that the amount that the instantaneous power is modulated (e.g., either up or down) can be based on a variety of methods. For example, controller 312 can send signals to laser source 306 to cause laser source 306 to modulate the instantaneous power of the laser pulses (e.g., laser pulse 404) based on a look up table comprising an indication of vapor bubble length versus pulse on-time (e.g., as a function of peak power, wavelength, or the like). As another example, controller 312 can send signals to laser source 306 to cause laser source 306 to modulate the instantaneous power of the laser pulses (e.g., laser pulse 404) based on a simulation or a model configured to predict or infer vapor bubble length based on lasing parameters (e.g., peak power, wavelength, on-time, or the like). With yet another example, controller 312 can send signals to laser source 306 to cause laser source 306 to modulate the instantaneous power of the laser pulses (e.g., laser pulse 404) based on directly measuring the length of the vapor bubble.

For example, controller 312 can send signals to laser source 306 to cause laser source 306 to turn pulsing on and off at regular intervals. As a specific example, generate laser pulses for 100 microseconds (μs) and pausing generation of laser pulses for 50 μs, repeated until the prescribed quantity of energy is generated by the laser source may limit the reach of the laser energy to approximately 2.5 millimeters (mm). For any given combination of pulse energy and distance threshold, the present disclosure can generate a predetermined modulation sequence, which can be applied by the controller 312.

FIG. 6 illustrates a method 600 for modulating laser pulses to control the size of a gaseous pathway created by vapor bubble when the laser pulses are emitted into a liquid medium. Method 600, like method 500, can be representative of some or all the operations that may be executed by one or more components, devices, or systems described herein, such as one or more portions of laser system 300. Furthermore, method 600 can be implemented by a medical device arranged to emit laser energy into a liquid medium (e.g., treatment environment 400) as part of using the medical device as part of a therapeutic procedure (e.g., lithotripsy). Although the laser system 300 and the treatment environment 400 are used when describing method 600, embodiments are not limited in this context and the method 600 can be implemented by a laser system having a different arrangement to laser system 300 or including different components than shown or described with respect to laser system 300.

As depicted, method 600 may begin at block 602. At block 602 “receive an indication of a distance between a distal end of a fiber optic cable and a target” an indication of a distance between a distal end of a fiber optic cable and a target is received. For example, controller 312 may receive an indication (e.g., from a measurement subsystem, from one of optical components 308, or the like) an indication of the distances 410, which as depicted in FIG. 4 is the distance between the distal end 402 and the target 406.

Continuing to decision block 604 “distance changed more than allowed?” a determination of whether the distance has changed more than an allowed amount. For example, controller 312 can determine whether the distance 410 (e.g., received at block 602) changed more than an allowed (or threshold) amount. In a specific example, the controller 312 can determine whether the distance 410 changed more than a threshold amount (change threshold) within a set period of time. With some examples, the threshold for an allowed change in distance can be 10%, 20%, 25%, or the like. With some examples, the threshold for an allowed change can be 10% within a second, 20% within a second, or 25% within a second. With some embodiments, at decision block 604, controller 312 can also determine whether the distal end of the fiber optic cable 304 is pointed at stone or tissue and can continue to block 606 based on a determination that the distal end of the fiber optic cable is pointed at stone or proceed to block 614 and end the treatment (or said differently, collapse the vapor tunnel) based on a determination that the distal end of the fiber optic cable is pointed at tissue.

From decision block 604, method 600 can continue to block 606 or can skip to done block 614 and end. Specifically, the method 600 can continue from decision block 604 to block 606 based on a determination at decision block 604 that the distance 410 has not changed more than the threshold amount while the method 600 can skip to done block 614 and end based on a determination at decision block 604 that the distance 410 has changed more than the threshold amount. At done block 614 “end treatment” the treatment protocol can be suspended or terminated and a controller 312 can send a control signal to laser source 306 to cause 306 to pause or terminate generation of laser pulses.

At block 606 “generate, at a laser source, a number of pulses of laser energy” several pulses of laser energy can be generated by a laser source. For example, laser pulses 404 can be generated at laser source 306. Continuing to block 608 “emit, from the fiber optic cable, the laser energy” the generated pulses of laser energy can be emitted from the fiber optic cable. For example, the laser pulses 404 can be emitted from the distal end 402 of the fiber optic cable 304.

Continuing to block 610 “modulate the instantaneous power of the number of pulses of laser energy such that when the laser energy is emitted from the fiber optic cable into a liquid medium, a gaseous pathway formed by the pulses of laser energy collapses within a threshold of the distance” the instantaneous power of the laser pulses is modulated such that a gaseous pathway formed when the laser pulses are transmitted into a liquid medium collapses within a threshold of the distance. For example, controller 312 can send signals (e.g., control signals, information elements, or the like) to laser source 306 to cause laser source 306 to modulate the instantaneous power of the generated laser pulses (e.g., laser pulses 404) to cause the gaseous pathway 408 to collapse within threshold distance 412 (e.g., the collapse threshold).

Continuing to decision block 612 “continue treatment?” a determination on whether to continue treatment can be made. For example, controller 312 can determine whether to continue the treatment protocol based on a received input (e.g., foot pedal actuation, or the like). As another example, controller 312 can determine whether to continue the treatment protocol based on a parameter of the protocol (e.g., total time emitting laser pulses, gross power of generated laser pulses, or the like). From decision block 612, method 600 can return to block 602 or can continue to block 614. Method 600 can return to block 602 from decision block 612 based on a determination at decision block 612 that the treatment protocol is to be continued while method 600 can continue to block 614 from decision block 612 based on a determination at decision block 612 that the treatment protocol is not to be continued. In such a manner, method 600 provides modulation of the laser pulses to be repeated in cycles to continue the treatment protocol while maintaining (e.g., growing or collapsing) the gaseous pathway 408 such that the gaseous pathway 408 is within a threshold from the distance 410, even where the distance 410 changes during the treatment protocol.

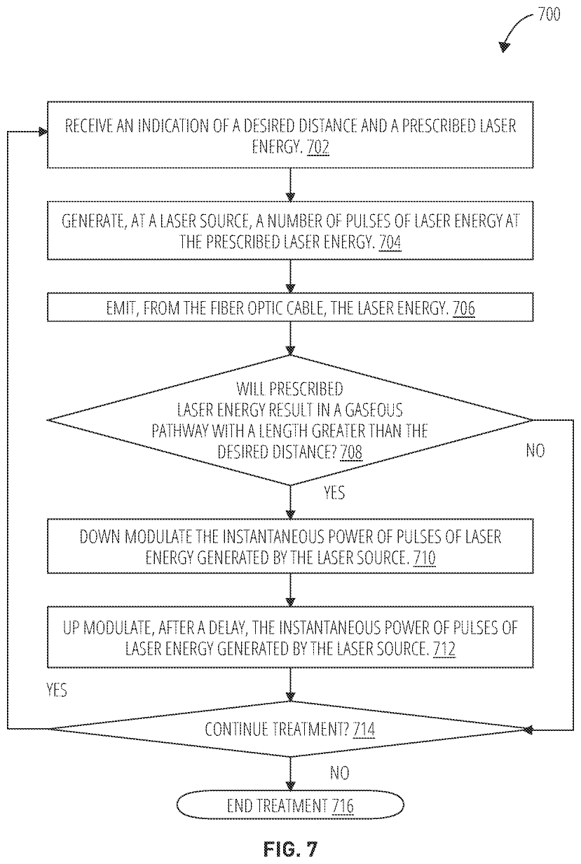

FIG. 7 illustrates a method 700 for modulating laser pulses to control the size of a gaseous pathway created by vapor bubbles when the laser pulses are emitted into a liquid medium. Method 700, like method 500 and 600, can be representative of some or all the operations that may be executed by one or more components, devices, or systems described herein, such as one or more portions of laser system 300. Furthermore, method 700 can be implemented by a medical device arranged to emit laser energy into a liquid medium (e.g., treatment environment 400) as part of using the medical device as part of a therapeutic procedure (e.g., lithotripsy). Although the laser system 300 and the treatment environment 400 are used when describing method 700, embodiments are not limited in this context and the method 700 can be implemented by a laser system having a different arrangement to laser system 300 or including different components than shown or described with respect to laser system 300.

As depicted, method 700 may begin at block 702. At block 702 “receive an indication of a desired distance and a prescribed laser energy” an indication of a desired distance (e.g., of laser propagation, between a distal end of a fiber tip and a target, of a vapor tunnel, or the like) and a prescribed laser energy is received. For example, controller 312 may receive an indication (e.g., from a user interface, or the like) an indication of a desired length of the gaseous pathway 408 (e.g., distance 410, or the like) as well as an indication of a desired or prescribed energy for laser pulses 404. Continuing to block 704 “generate, at a laser source, a number of pulses of laser energy” several pulses of laser energy can be generated by a laser source. For example, laser pulses 404 can be generated at laser source 306. Continuing to block 706 “emit, from the fiber optic cable, the laser energy” the generated pulses of laser energy can be emitted from the fiber optic cable. For example, the laser pulses 404 can be emitted from the distal end 402 of the fiber optic cable 304.

Continuing to decision block 708 “will prescribed laser energy result in a gaseous pathway with a length greater than the desired distance?” a determination of whether the prescribed laser energy (e.g., of pulses generated as block 704, or the like) will result in a gaseous pathway 408 greater than the desired distance. In a specific example, the controller 312 can determine whether the energy of pulses 404 will cause gaseous pathway 408 to grow to a distance 410 greater than the desired distance. From decision block 708, method 700 can continue to block 710 or can skip to block 714. Specifically, the method 700 can continue from decision block 708 to block 710 based on a determination at decision block 708 that the laser energy will result in a distance 410 associated with the gaseous pathway 408 formed by pulses generated at block 704 and emitted at block 706 to grow longer than the desired distance while the method 700 can skip to block 714 based on a determination at decision block 708 that the laser energy will not result in a distance 410 associated with the gaseous pathway 408 formed by pulses generated at block 704 and emitted at block 706 to grow longer than the desired distance.

At block 710 “down modulate the instantaneous power of pulses of laser energy generated by the laser source” the laser energy of pulses generated by the laser source can be modulated down. For example, the instantaneous power of the pulses of laser energy can be modulated down a specified percentage (e.g., 10%, 20%, 25%, etc.) so that the gaseous pathway 408 collapses. As a specific example, controller 312 can determine do modulate the instantaneous power of the pulses of laser energy down a specified percentage (e.g., 10%, or the like) after a time corresponding to an on-time for pulses at the prescribed energy that is associated with a gaseous pathway of length equal to the desired distance.

Continuing to block 712 “up modulate, after a delay, the instantaneous power of pulses of laser energy generated by the laser source” the laser energy of pulses generated by the laser source can be modulated up. For example, the instantaneous power of the pulses of laser energy can be modulated back up to the prescribed power so that the gaseous pathway will again grow to the desired length. With some examples, at block 712, controller 312 can modulate the instantaneous power of the laser pulses back up to the prescribed power after a delay (e.g., 5 microseconds, 10 microseconds, 20 microseconds, or the like).

It is to be appreciated, for long pulses, this procedure outlined in method 700 can be repeated several times. For example, method 700 can include decision block 714 “continue treatment?” where a determination on whether to continue treatment can be made. For example, controller 312 can determine whether to continue the treatment protocol based on treatment parameters (e.g., total energy delivered to the target, total on-time, or the like). From decision block 714, method 700 can return to block 702 or can continue to block 716 and end. That is, method 700 can return to block 702 from decision block 714 based on a determination at decision block 714 that the treatment protocol is to be continued while method 700 can continue to block 716 from decision block 714 based on a determination at decision block 714 that the treatment protocol is not to be continued.

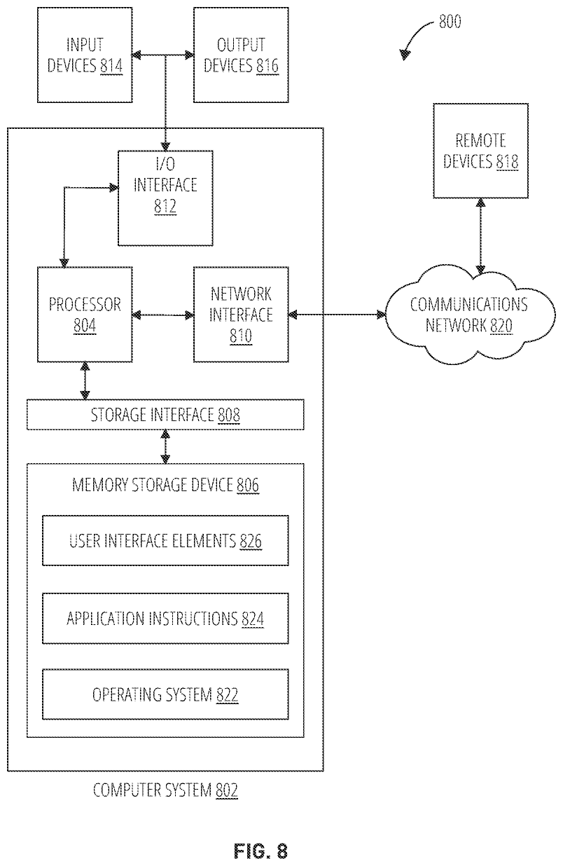

FIG. 8 is a block diagram of a computing environment 800 including a computer system 802 for implementing embodiments consistent with the present disclosure. In some embodiments, the computing environment 800, or portion thereof (e.g., the computer system 802) may comprise or be comprised in a laser system (e.g., the controller 312 of the laser system 300 can embody portions of the computing environment 800). Accordingly, in various embodiments, computer system 802 may determine a distance and control modulation of power of laser pulses based on the determined distance to cause a gaseous pathways formed when the laser pulses are emitted into a liquid medium to collapse within a threshold of the distance.

The computer system 802 may include a central processing unit (“CPU” or “processor”) 804. The processor 804 may include at least one data processor for executing instructions and/or program components for executing user or system-generated processes. A user may include a person, a person using a device such as those included in this disclosure, or another device. The processor 804 may include specialized processing units such as integrated system (bus) controllers, memory management control units, floating point units, graphics processing units, neural processing units, digital signal processing units, etc. The processor 804 may be disposed in communication with input devices 814 and output devices 816 via I/O interface 812. The I/O interface 812 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n /b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 812, computer system 802 may communicate with input devices 814 and output devices 816. In some embodiments, the processor 804 may be disposed in communication with a communications network 820 via a network interface 810. In various embodiments, the communications network 820 may be utilized to communicate with a remote memory storage device 806, such as for accessing look-up tables, performing updates, or utilizing external resources. The network interface 810 may communicate with the communications network 820. The network interface 810 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 820 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 820 may include a variety of network devices, including routers, bridges, servers, computing devices, storage devices, etcetera. In some embodiments, the processor 804 may be disposed in communication with a memory storage device 806 via a storage interface 808. The storage interface 808 may connect to memory storage device 806 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 806 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 806 may store a collection of program or database components, including, without limitation, an operating system 822, an application instructions 824, and a user interface elements 826. In various embodiments, the operating system 822 may facilitate resource management and operation of the computer system 802. Examples of operating systems include, without limitation, APPLE® MACINTOSH® OS X®, UNIX®, UNIX-like system distributions (E.G., BERKELEY SOFTWARE DISTRIBUTION® (BSD), FREEB SD®, NETBSD®, OPENB SD, etc.), LINUX® DISTRIBUTIONS (E.G., RED HAT®, UBUNTU®, KUBUNTU®, etc.), IBM® OS/2®, MICROSOFT® WINDOWS® (XP®, VISTA®/7/8, 10 etc.), APPLE® IOS®, GOOGLE™ ANDROID™, BLACKBERRY® OS, or the like.

The application instructions 824 may include instructions that when executed by the processor 804 cause the processor 804 to perform one or more techniques, steps, procedures, and/or methods described herein, such to control a length of a gaseous pathway (e.g., gaseous pathway 408). For example, application instructions 824, when executed by processor 804 can cause processor 804 to perform the method 500 and/or the method 600.

The user interface elements 826 may facilitate display, execution, interaction, manipulation, or operation of program components through textual or graphical facilities. For example, user interfaces may provide computer interaction interface elements on a display system operatively connected to the computer system 802, such as cursors, icons, checkboxes, menus, scrollers, windows, widgets, etcetera. The user interface elements 826 may be employed by application instructions 824 and/or operating system 822 to provide, for example, a user interface with which a user can interact with computer system 802. In some embodiments, the user interface elements 826 may be integrated with the display (e.g., display 314).

With respect to the use of substantially any plural and/or singular terms herein, those having skill in the art can translate from the plural to the singular and/or from the singular to the plural as is appropriate to the context and/or application. The various singular and/or plural permutations are expressly set forth herein for sake of clarity and not limitation.

It will be understood by those within the art that, in general, terms used herein, and are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended. For example, as an aid to understanding, the detail description may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to disclosures containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

All the devices and/or methods disclosed and claimed herein can be made and executed without undue experimentation considering the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope and concept of the disclosure as defined by the appended claims.

What is claimed is:

1. A system, comprising:
a laser source; and
a controller coupled to the laser source, the controller comprising a processor and memory, the memory comprising instructions that when executed by the processor cause the system to:
generate, at the laser source, a plurality of pulses of laser energy, the plurality of pulses of laser energy to be emitted from a fiber optic cable disposed in a liquid environment; and
modulate the instantaneous power of the plurality of pulses of laser energy to cause a gaseous pathway in the liquid environment to collapse within a threshold of a specified distance.

2. The system of claim 1, wherein the plurality of laser pulses form vapor bubbles in the liquid medium and wherein the gaseous pathway is formed by the vapor bubbles.

3. The system of claim 2, wherein the instantaneous power is modulated with an opposite phase to the growth of the vapor bubbles.

4. The system of claim 3, wherein the instantaneous power is modulated to a lower level at a time estimated for the vapor bubble to reach a maximal size.

5. The system of claim 1, the instructions when executed by the processor further cause the system to:
receive an indication of a distance between an end of the fiber optic cable and a target, wherein the distance is the specified distance; and
modulate the instantaneous power of the plurality of pulses of laser energy based on the distance, the initial power of the plurality of pulses of laser energy, and an absorption coefficient of the liquid at a wavelength of the laser source.

6. The system of claim 1, the instructions when executed by the processor further cause the system to:
receive an indication of an updated distance between the end of the fiber optic cable and the target; and
modulate the instantaneous power of the plurality of pulses of laser energy to cause the gaseous pathway in the liquid environment to collapse within the threshold of the updated distance.

7. The system of claim 6, the instructions when executed by the processor further cause the system to:
determine whether an absolute difference between the distance and the updated distance is greater than or equal to a second threshold; and
terminate generation of the plurality of pulses of laser energy based on a determination that the absolute difference is greater than or equal to the second threshold.

8. The system of claim 1, wherein the threshold is less than or equal to the specified distance times 0.3.

9. The system of claim 8, wherein the threshold is greater than or equal to 0.

10. The system of claim 1, wherein the laser source is a Thulium fiber laser.

11. The system of claim 1, comprising the fiber optic cable, wherein the fiber optic cable is arranged to be inserted through a working channel of a ureteroscope.

12. A method, comprising:
generating, at a laser source, a plurality of pulses of laser energy, the plurality of pulses of laser energy to be emitted from a fiber optic cable disposed in a liquid environment; and
modulating the instantaneous power of the plurality of pulses of laser energy to cause a gaseous pathway in the liquid environment to collapse within a threshold of a specified distance.

13. The method of claim 12, wherein the plurality of laser pulses form vapor bubbles in the liquid medium and wherein the gaseous pathway is formed by the vapor bubbles.

14. The method of claim 13, wherein the instantaneous power is modulated with an opposite phase to the growth of the vapor bubbles.

15. The method of claim 14, wherein the instantaneous power is modulated to a lower level at a time estimated for the vapor bubble to reach a maximal size.

16. The method of claim 12, comprising:
receiving an indication of a distance between an end of the fiber optic cable and a target, wherein the distance is the specified distance; and
modulating the instantaneous power of the plurality of pulses of laser energy based on the distance, the initial power of the plurality of pulses of laser energy, and an absorption coefficient of the liquid at a wavelength of the laser source.

17. The method of claim 12, comprising:
receiving an indication of an updated distance between the end of the fiber optic cable and the target; and
modulating the instantaneous power of the plurality of pulses of laser energy to cause the gaseous pathway in the liquid environment to collapse within the threshold of the updated distance.

18. The method of claim 17, comprising:
determining whether an absolute difference between the distance and the updated distance is greater than or equal to a second threshold; and
terminating generation of the plurality of pulses of laser energy based on a determination that the absolute difference is greater than or equal to the second threshold.

19. The method of claim 12, wherein the threshold is less than or equal to the specified distance times 0.3.

20. The method of claim 4, wherein the threshold is greater than or equal to 0.
